(19) United States
(12) Patent Application Publication
(10) Pub. No.: US 2015/0164471 A1
(43) Pub. Date: Jun. 18, 2015

(54) ULTRASOUND CONDUCTIVE MEDIUM WITH LOCKING ELEMENT

(71) Applicant: G. Chad MORGAN, Chattanooga, TN (US)

(72) Inventor: G. Chad MORGAN, Chattanooga, TN (US)

(21) Appl. No.: 14/577,397

(22) Filed: Dec. 19, 2014

Related U.S. Application Data
(63) Continuation-in-part of application No. 14/096,641, filed on Dec. 4, 2013.
(60) Provisional application No. 61/919,233, filed on Dec. 20, 2013.
(60) Provisional application No. 61/765,361, filed on Feb. 15, 2013.
(60) Provisional application No. 61/792,909, filed on Mar. 15, 2013.
(60) Provisional application No. 62/077,584, filed on Nov. 10, 2014.

(51) Int. Cl. A61B 8/00

(52) U.S. Cl. CPC A61B 8/4281; A61B 8/4455

(57) ABSTRACT

Embodiments associated with an ultrasound conductive medium are described. In one embodiment, an ultrasound conductive substance is formed into a solidified shape and includes a locking element for connecting to a device.

### CROSS REFERENCE TO RELATED APPLICATIONS

This patent disclosure claims the benefit of U.S. Provisional Patent Application Ser. No. 61/919,233 entitled “Gel with locking element and gel adaptor” filed on Dec. 20, 2013, inventor G. Chad Morgan, which is hereby wholly incorporated by reference in its entirety.

This patent disclosure claims the benefit of and is a Continuation-In-Part of U.S. application Ser. No. 14/096,641 entitled “Ultrasound device with cavity for conductive medium, filed on Dec. 4, 2013, inventor G. Chad Morgan, which claims the benefit of U.S. Provisional Patent Application with Ser. No. 61/765,361 filed on Feb. 15, 2013, entitled “Ultrasound device with recessed diaphragm,” and U.S. Provisional Patent Application with Ser. No. 61/792,909, filed Mar. 15, 2013, entitled “Alternating frequency ultrasound device.”

This patent disclosure claims the benefit of U.S. Provisional Patent Application Ser. No. 62/077,584 entitled “Attachable adaptor with locking mechanism for ultrasound conductive medium” filed on Nov. 10, 2014, inventor G. Chad Morgan, which is hereby wholly incorporated by reference in its entirety.

### BACKGROUND

In the medical field, ultrasound devices operate with frequencies from 20 kHz up to several gigahertz. Ultrasound devices may be used for therapeutic procedures by stimulating the tissue beneath the skin's surface using very high frequency sound waves and/or for generating images of internal structures.

Ultrasound is applied using a device that includes a transducer or applicator that is in contact with a patient's skin. Liquid gel is used on all surfaces of the device's head to reduce friction and assist transmission of the ultrasonic waves. The liquid gel is squeezed out of a bottle and spread over the patient's skin. Since the gel is a liquid, the gel is difficult to contain within a desired area of the skin and the thickness of the gel cannot be controlled. Lack of consistent and desired thickness of the gel leads to a less than optimal ultrasound application. When the ultrasound procedure is completed, the patient is required to wipe off the gel from the patient's skin. Typically, the gel is not completely removed and the cleaning process is uncomfortable.

Additionally, liquid gel has been stored in large containers that are typically heated. Heating the gel makes it more comfortable for a patient when the gel is applied. However, the heat and long term storage of such containers creates issues with sterility.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIGS. 1A and 1B illustrate one embodiment of a preconfigured gel from a top view and side view, respectively.

FIG. 1C illustrates another embodiment of the preconfigured gel from a side view.

FIG. 1D illustrates a 3-dimensional image of the preconfigured gel shown in two sizes.

FIG. 2A illustrates one embodiment of an ultrasound device shown with components unassembled.

FIG. 2B illustrates the ultrasound device of FIG. 2A in an assembled view.

FIGS. 3A-3D illustrate various views of one embodiment of a diaphragm.

FIG. 4A shows the gel piece 140 being inserted into a cavity of the diaphragm.

FIG. 4B shows the gel piece 140 in an inserted state in the cavity of the diaphragm.

FIGS. 5A, 5B, 5C, and 5D illustrate top, perspective, and side views of another embodiment of a preconfigured gel configured with a locking ring.

FIG. 5E illustrates a top view of another embodiment of the gel with a tabs as a locking element.

FIG. 5F illustrates a side view of FIG. 5E.

FIG. 5G illustrates a top view of another embodiment of a rectangular solidified gel with a tabs as a locking element.

FIG. 5H illustrates a side view of FIG. 5G.

FIGS. 6A, 6B, 6C, and 6D illustrate various views of one embodiment of an adaptor configured with a locking ring/recess for the preconfigured gel shown in FIGS. 5A-5C.

FIG. 6E illustrates a cross-section view of the adaptor through section A-A of FIG. 6B.

FIGS. 7A-7E illustrate multiple views of another embodiment of an attachable adaptor including a locking mechanism that can be used with solidified gel of FIGS. 5A-5D.

### DETAILED DESCRIPTION

Embodiments of an ultrasound conductive medium are disclosed herein that may be used with ultrasound devices and/or ultrasound procedures.

In one embodiment, a conductive medium is formed into a solidified substance that is preconfigured into a predefined shape (herein also referred to as a “gel piece”). The predefined shape may be made to correspond to a recessed cavity of an ultrasound device or to cavity of a gel adaptor that attaches to the head of the ultrasound device. In one or more embodiments, the gel piece includes a locking element for securing the gel piece to a component of an ultrasound device (e.g., the head portion or gel adaptor).

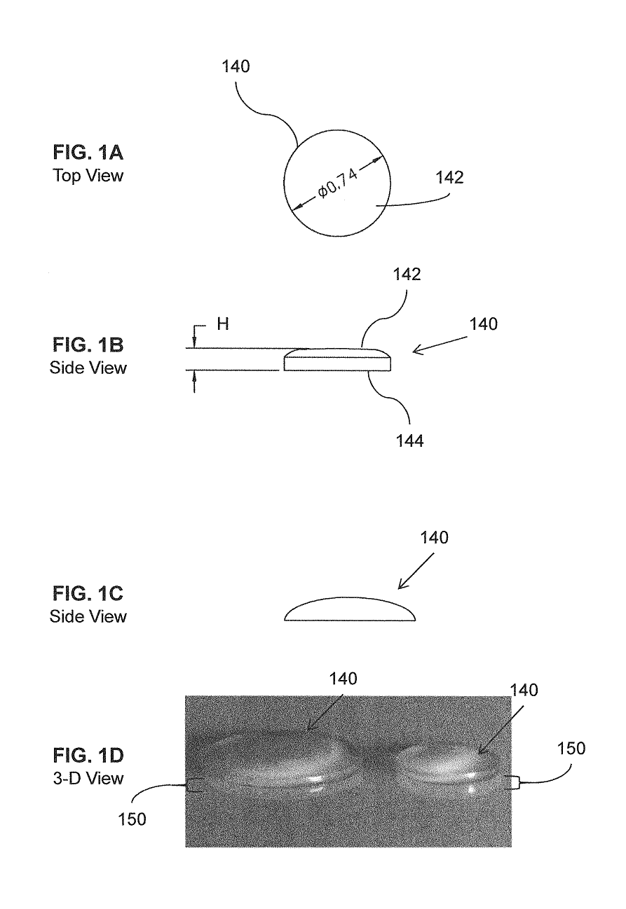

With reference to FIG. 1A and 1B, one embodiment of preconfigured gel 140 is illustrated. FIG. 1A shows a top view of the gel 140 and FIG. 1B shows a side view. FIG. 1C illustrates a side view of another embodiment of the gel 140 with a different shape. FIG. 1D illustrates a 3-dimensional image of the preconfigured gel 140 shown in two sizes where the gel 140 represents the shape of FIGS. 1A and 1B. The gel 140 is shown placed on top of a base 150 from which the gel 140 is removed prior to use. The base 150 is not required.

In one embodiment, the preconfigured gel 140 (or gel piece) is formed with a circular top surface 142 and bottom surface 144 and has a general configuration of a disk. The gel 140 includes a thickness or height H. In one embodiment, the height H of the gel 140 is about 0.15 inches and the diameter is about 0.74 inches. In another embodiment, the diameter may be about 1.17 inches. In one embodiment, the height H is less than one inch. For larger ultrasound heads or devices, the gel 140 may be made larger to fit the head, where the head would include a larger gel cavity.

The shape and size of the gel piece 140 may be defined to correspond to a gel receiving cavity of an ultrasound device in which the gel piece 140 is to function with. Of course, the gel piece 140 may be implemented in other shapes and sizes (e.g., 3-dimensional oval or polygon, other shapes with curved sides).

The top surface 142 of the disk shape may be flat (see FIG. 1B) or curved (see FIG. 1C) in order to provide good contact with a patient's skin. In other embodiments, the gel piece 140 may be formed with irregular shapes such as having the top surface 142 that is larger than the bottom surface 144 (e.g., mushroom shape).

In one embodiment, the preconfigured gel (gel piece) 140 is a device or an article of manufacture. The gel piece 140 is formed from an aqueous material that functions as a conductive medium and coupling medium for ultrasound energy. The aqueous material is for example a liquid or jelly-like substance/composition that is molded into a desired shape and processed to retain its shape as a solid form. Various processes may be used such as curing, heating, cooling, or other process that can solidify the substance based on the gel composition used and desired specifications. For example, the gel composition is processed to solidify the gel to a desired extent so that its shape is set. In different embodiments, the preconfigured gel can be solidified to different degrees or ranges as desired such as being a soft and flexible object, being a rigid object, or any state in between (e.g., semi-rigid and/or flexible structure).

In one embodiment, the gel piece 140 is formed to retain its shape and have memory. Once the shape is set, which has a predetermined amount of gel, the preconfigured gel provides a consistent amount and thickness of gel for an ultrasound procedure. The gel piece 140 is formed in advance so that it is ready for use with an ultrasound device and provides a premeasured and consistent amount of gel. After being used in an ultrasound procedure, the preconfigured gel 140 is removed from the ultrasound device and replaced with a new piece of preconfigured gel.

As such, the amount of gel used during a scan is fully controlled by the piece of preconfigured gel. Furthermore, the preconfigured gel provides for greater sterility because pieces of gel can be packaged individually to prevent contamination. Dispensing and applying liquid gel on a patient in a random and immeasurable manner is eliminated. Furthermore, using a controlled amount of gel reduces the amount of gel needed for a scan, which can reduce the cost of using gel.

In one embodiment, the preconfigured gel 140 may be formed or molded into a desired shape using a variety of molding or manufacturing processes. One process may involve using molds. Multiple molds can be used to create multiple pieces of the preconfigured gel at a time. For example, a tray of patterned shapes can be used where the gel composition in a liquid form is inserted into each shape of the tray. The gel composition is then processed to solidify the gel to a desired extent so that its shape is set (e.g., the composition holds its shape, does not flow as a liquid). Of course, other processes may be used to create the preconfigured gel such as injection molding, 3-D printing, and so on.

Ultrasound Device Embodiment

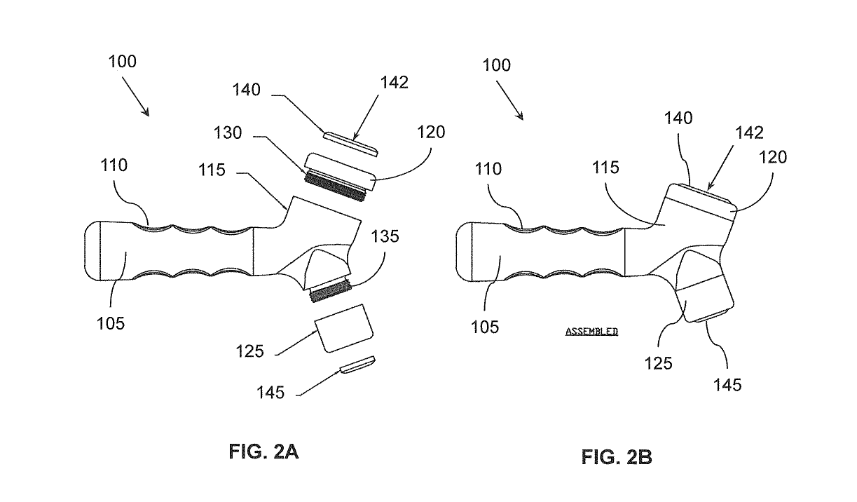

With reference to FIGS. 2A and 2B, in one embodiment, an ultrasound device 100 is described that may function with the gel piece 140.

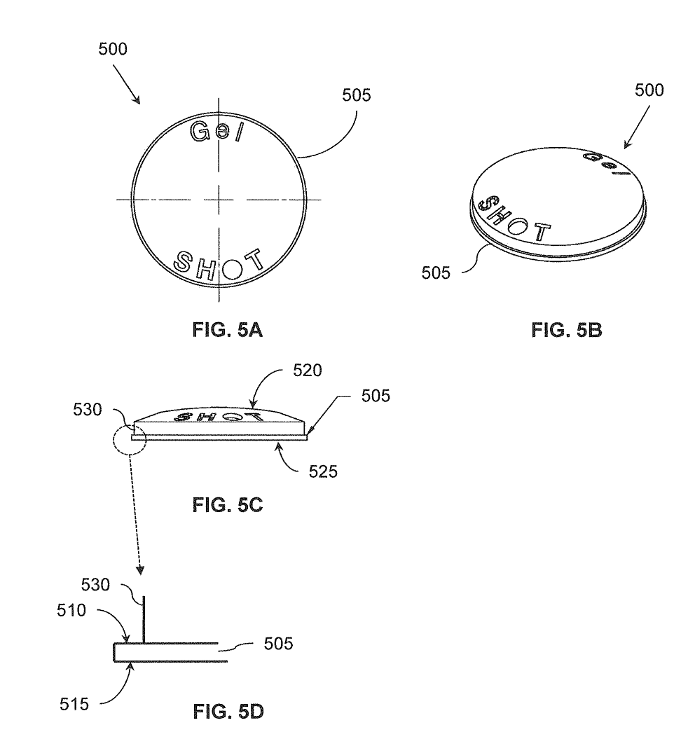

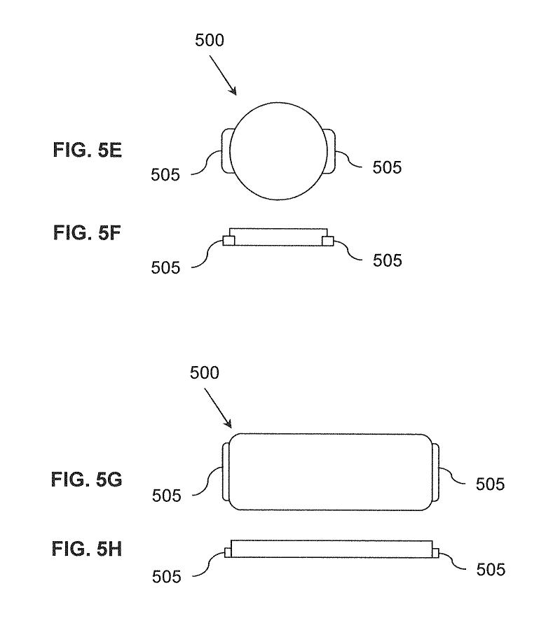

Overall, the devices shown in FIGS. 2A-2B, 3A-3D, 4A-4B, 6A-6E, and 7A-7E are used as examples to show how the gel piece 140 (and gel piece 500 in FIG. 5A-5C) may be used in practice and to provide context. Of course, other types of devices may be used with the gel pieces described herein.

With reference to FIG. 2A, the ultrasound device 100 includes a diaphragm/head 120 configured with a recessed cavity for receiving and containing the preconfigured gel piece 140. In another embodiment, the diaphragm is configured as the head or nose of an ultrasound device, probe, or applicator where the diaphragm includes an ultrasound transducer (e.g., piezoelectric crystal) and a cavity for containing the preconfigured gel 140. In some embodiments, the diaphragm/head is integral with the body of the ultrasound device 100 or is attachable and detachable to be replaceable.

FIG. 2A illustrates the ultrasound device 100 in a partially unassembled state and FIG. 2B shows the device 100 in an assembled state. The device 100 is configured as a hand-held device including an elongated handle 105 that may include one or more finger grips 110 (e.g., indentations, ridges, and so on). The handle 105 is connected to a head 115 that includes one or more sides that connect to a diaphragm. In the illustrated embodiment, the head 115 includes two sides where the first side includes diaphragm 120 and the second side includes diaphragm 125. Of course, other types of ultrasound devices or probes may be used with the preconfigured gel described herein. For example, the ultrasound device may be a single head device that is circular or rectangular in shape and/or may have a flat or arced surface for holding the gel piece. Thus the embodiment of FIGS. 2A-2B are not intended to be limiting.

In one embodiment, diaphragm 120 is configured with a connector 130 that is threaded to insert and connect with a corresponding threaded socket in the head 115. Similarly, the second side of the head 115 may include a connector 135 that is threaded for connecting with a threaded socket within the diaphragm 125. In another embodiment, the connectors 130 and 135 may be configured as a quick-connect/disconnect device so that the diaphragms 120 and 125 can be connected by pushing and snapping into place or disconnected by pulling off with a small amount of force. Thus, in one embodiment, the diaphragms 120 or 125 are configured as replaceable components. In another embodiment, the head 115 and diaphragm 125 may be integral with each other.

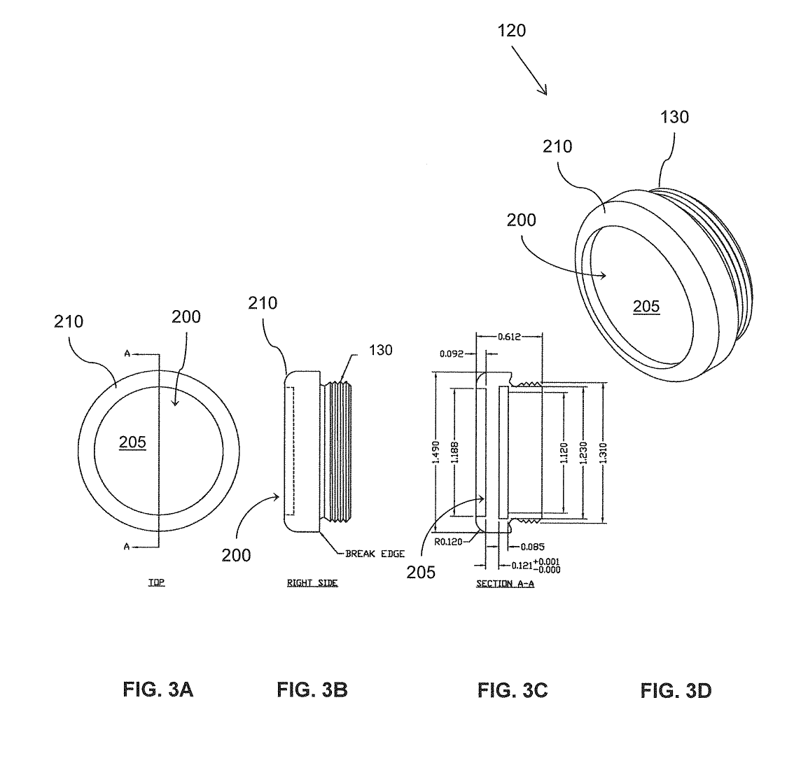

With reference to diaphragm 120, the diaphragm includes a recessed cavity (shown in FIG. 3D) that is configured to receive and contain a conductive medium 140 used during an ultrasound scan. In one embodiment, the conductive medium 140 is a portion of gel (e.g., gel piece, gel pad) that is preconfigured to fit into the cavity, as seen in FIG. 2B where gel 140 is inserted into the diaphragm 120. Likewise, diaphragm 125 includes a cavity to receive a conductive medium 145 when used during a scan. The diaphragm 120 is explained in more detail with reference to FIG. 2.

With reference to FIG. 3A-D, one embodiment of diaphragm 120 is shown in a Top View FIG. 3A, Right Side View FIG. 3B, Cross-Section View FIG. 3C through A-A, and a Perspective View FIG. 3D.

In one embodiment, the diaphragm 120 a housing formed from metal, metallic, or other conductive material that functions with ultrasound energy. The diaphragm 120 includes a cavity 200 that is configured to receive a conductive medium. The cavity 205 is defined by inside surface 205 and a sidewall 210. The surface 205 is generally a circular shape but other shapes may be used.

In one embodiment, the sidewall 210 is a continuous edge or rim around the perimeter of the surface 205. In another embodiment, the sidewall 205 may include one or more notches (not shown). A notch may be used remove a preconfigured piece of gel from within the cavity 200 by inserting a finger in the notch to access the gel within the cavity. In another embodiment, the sidewall 205 may be perforated or be configured as two or more portions such as prongs that can hold a piece of solid gel (e.g., a gel piece 140 shown in FIG. 1A and 1B, gel piece 500, etc.).

The cavity 205 is configured as a containment area for receiving a conductive preconfigured gel (e.g., a gel piece). In one embodiment as explained previously, the conductive medium is configured to correspond to the shape of the cavity 205. A gel piece can be inserted into the cavity 200 where the gel is held in place by at least surface tension with the surface 205 and/or friction with the inside surface of the sidewall 210. In this manner, the diaphragm 120 self-contains the gel to be used during a scan. When a scan is complete, the gel piece 140 is simply removed and another piece of gel can be inserted for a subsequent scan.

In one embodiment, in the inside of the diaphragm 120, the diaphragm 120 includes a transducer (e.g., piezoelectric crystal) (not shown) for generating ultrasound waves. The transducer is connected to the diaphragm within the threaded shaft 130 and secured against inside surface of the surface 205 (e.g., on the back side of surface 205).

FIG. 3C illustrates a cross-section view through A-A. The various dimensions shown (e.g., in inches) are only exemplary of one embodiment. It is not intended to limit the construction of the diaphragm shown since the diaphragm can be formed with different shapes and sizes.

Device Components

Details of the ultrasound device 100 are beyond the scope of the gel piece 140 and thus are only described generally. With reference again to FIG. 2A, the handle 105 and head 115 are formed from a housing that may contain one more components (not shown) configured to generate and detect ultrasound energy. In one embodiment, the device 100 includes an energy generating module operative to generate a driving signal that can be transformed into ultrasonic energy. The energy generating module includes a local power source or receives power from a remote source via a power cord, an oscillator, and a driver component.

The portable ultrasound device 100 also includes an ultrasound transducer having a piezoelectric or other electric component. The ultrasound transducer is operative to receive the driving signal from the energy generating module and transforms the driving signal into ultrasonic energy. There are many different types of internal components that can be used to implement the ultrasound device. Since they are not the focus of the present disclosure, they are not described in detail.

In another embodiment, the device 100 may include an internal memory for storing ultrasound data collected by the device 100. The device 100 may include an interface for communicating the data from the memory to a remote device. The device 100 can be configured to communicate the data via a wire connection and/or a wireless connection.

Solidified Gel Piece Embodiment

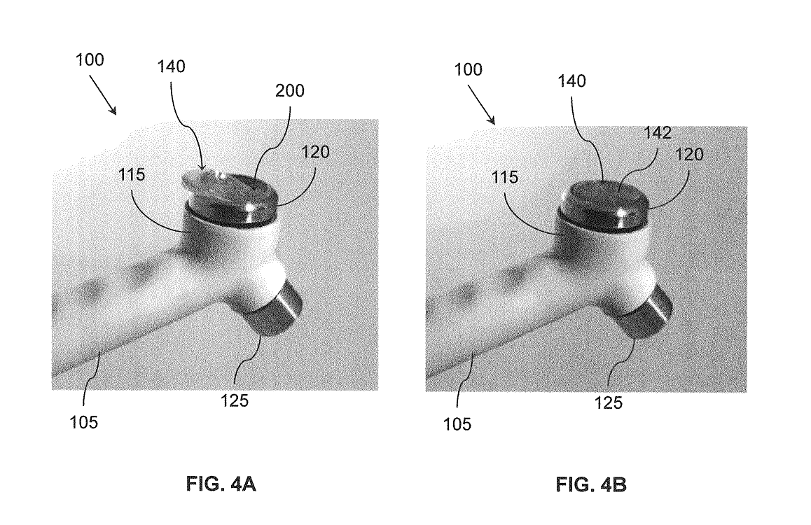

With reference to FIG. 4A, in another embodiment, the gel piece 140 is a conductive medium for use with therapeutic ultrasounds, imaging ultrasounds and electrotherapy devices. The gel piece 140 is made from a substance (e.g. gel-based, water-based) that conducts ultrasound energy and is solidified into a predefined shape. In one embodiment, the conductive medium is preconfigured in a pad or disk shape that fits into and is contained within the cavity 200 of the diaphragm 120 or head 115. In one embodiment, the gel piece 140 is configured to fit into and generally correspond to the shape of the cavity 200. Of course, other shapes can be used based on the shape and configuration of the cavity 200 of the diaphragm 120. The gel piece 140 is preformed to a desired shape and can be inserted and removed from the ultrasound device 100 when needed.

In FIG. 4A, the gel piece 140 is shown being inserted into the cavity 200 of the diaphragm 120. FIG. 4B shows the gel piece 140 in an inserted state in the cavity 200 of the diaphragm 120. Once inserted into the cavity 200, an exterior surface (e.g., top surface 142) of the gel 140 extends out from the diaphragm so that the gel 140 contacts the skin of a patient (see FIG. 2B) to minimize air pockets between the skin and the transducer (not shown) in the diaphragm 120. In one embodiment, the gel 140 functions as a standoff between a patient's skin and the diaphragm 120. One purpose of the gel is to convey acoustic energy from the ultrasound head to the tissue of a patient without passing through air. Air can interfere or degrade the ultrasound signals.

Thus during a scan, the gel piece 140 moves with the ultrasonic device by being a part of the diaphragm 120 or covering the diaphragm.

Spreading gel on a patient is eliminated. In one embodiment, the gel is maintained within the cavity 200 by friction between the gel and the surfaces of the diaphragm. In one embodiment, the gel is maintained over and covering the head or diaphragm of the ultrasound device.

As one example of dimensions, which is not intended to be limiting, the height H of the gel piece 140 is constructed to be slightly greater than the height of the sidewall 210 that defines the cavity 200 (see FIG. 3B and 3C). For example, the height H of the gel piece 140 may be about 0.15 inches and the height of the sidewall 210 may be about 0.092 inches. Of course, other relationships of size can be implemented.

Air and other gases may impede sound waves. In one embodiment, the gel 140 is a solid piece of gel yet flexible. The gel 140 prevents the formation of air bubbles between the transducer and the patient's skin and helps conduct sound waves from the transducer into the patient's body.

Solidified Gel with Locking Element Embodiment

FIGS. 5A, 5B, and 5C illustrate top, perspective, and side views, respectively of another embodiment of a device or article of manufacture that is made from an ultrasound conductive substance and is formed into a solidified shape (e.g., preconfigured gel 500). In general, solidified gel 500 is another embodiment of the solidified gel piece 140 and is generally disk-shaped.

The preconfigured gel 500 is configured with a locking element 505. The locking element 505 is generally a protruding portion of gel that extends out from the solidified gel surface. The locking element 505 functions to secure and/or restrict movement of the gel 500 when inserted into a component of an ultrasound device. For example, the component of the ultrasound device may be a gel cavity in the head portion of an ultrasound device (see device 100, head portion 120) or a gel cavity of an adaptor device that holds solidified gel and is attachable to the head portion of an ultrasound device.

In one embodiment, the side wall of the solidified gel 500 is formed with a locking element 505 that is a tab that extends out from the sidewall and extends around the perimeter of the gel 500. Since the tab extends around the perimeter of the gel 500, the tab is generally a ring-shaped tab. From a different perspective, the locking element 505 may be formed by having the base of the gel have a larger diameter that the top portion of the gel 500 as seen in the top view of FIG. 5A. However, the locking element 505 may be positioned along any desired location along the height of the sidewall.

In one embodiment, the locking element 505 is integral with the body of the solidified gel 500 and made from the same substance as a one piece article (e.g., a single unitary piece of solidified gel). The solidified gel 500 and locking element 505 may be formed/manufactured as in the previously described molding process for the gel piece 140. In another embodiment, the locking element 505 may be separately attached to the gel body 500 to add the locking element.

In other embodiments, rather than being one continuous tab/ring around the perimeter, the locking element 505 may be configured as one or more partial tabs where each partial tab is less than the circumference/perimeter of the gel 500. In another embodiment, the locking element 505 may be configured as one or more strips of solidified gel formed as tabs, ribs, lips, edges, or other protrusions that extend out from the sidewall of the gel body in horizontal and/or vertical directions and may have any desired shape. In general, FIG. 5C shows the locking element 505 formed in a horizontal direction relative to the body of the gel 500 (e.g., generally parallel to the bottom surface 525 and extending out from the bottom surface 525). Thus, a vertical protrusion/rib/tab would extend along the side wall 530 in a direction between the top surface 520 of the gel 500 and the bottom surface 525 (e.g., generally perpendicular to the bottom surface 525).

In one embodiment, the solidified shape of gel 500 is generally disk-shaped as seen in FIGS. 5A-5B. The disk shape may include a flat or curved top surface 520 (that contacts the patient) and a flat bottom surface 525.

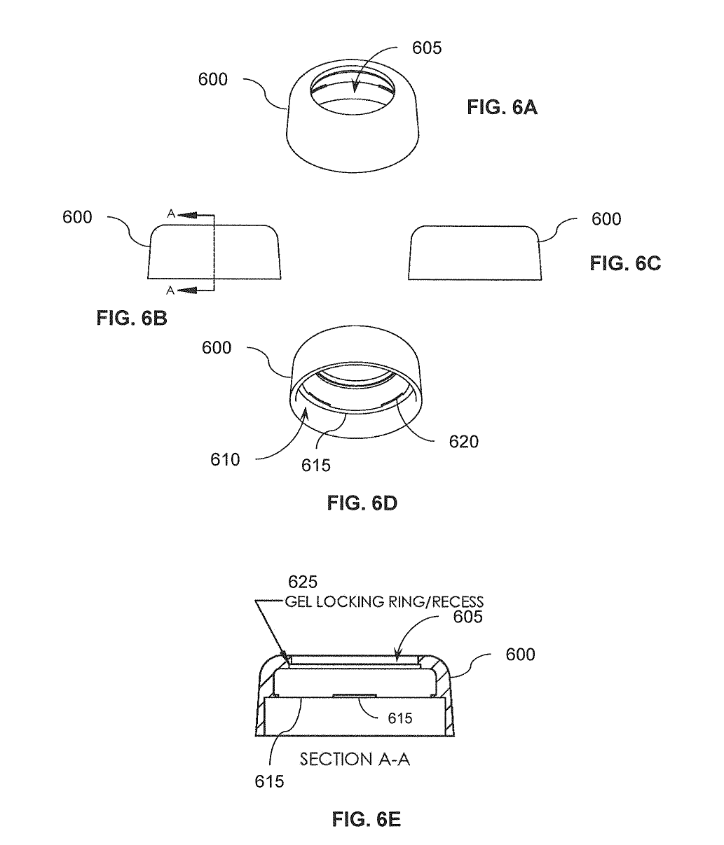

In one embodiment, the locking element 505 is configured to fit into a corresponding locking recess in the head of an ultrasound device or in a gel adaptor (see FIGS. 6A-6E). When the gel 500 is inserted into a gel cavity in the head (e.g., in the diaphragm 120 (FIG. 3D), in adaptor 600 (FIG. 6A), or similar cavity), the gel 500 is restricted from unintentionally falling out of the gel cavity since the locking element 505 interconnects with (is fitting within) a recess or edges in the wall of the gel cavity.

FIG. 5D shows an expanded view of a portion of the locking element 505 and side wall from FIG. 5C. The locking element 505 extends out from the side of the solidified shape of the gel 500 and includes two opposing side surfaces 510 and 515. These surfaces allow edges or walls from the gel cavity of the ultrasound device or adapter to interconnect with, or clamp down on the locking element 505 and secure the gel 500. In FIG. 5D, the bottom surface 515 of the tab 505 is integrated with the bottom surface 525 of the gel body 500.

FIGS. 5E-5H illustrate other embodiments of the solidified gel 500 with a locking element 505 that is configured as multiple tabs/protrusions that extend out the side wall of the disk-shaped body. FIG. 5E illustrates a top view of another embodiment of the gel 500 with a tabs 505 as a locking element formed on opposite sides of the gel 500. FIG. 5F illustrates a side view of gel 500 from FIG. 5E where tabs 505 are less than the height of the gel body 500.

FIG. 5G illustrates a top view of another embodiment of a rectangular solidified gel 500 with tabs 505 as locking elements. The rectangular body is regarded as a disk-shape that is elongated. The elongated body is flexible to bend and lay on an elongated curved transducer head of an ultrasound device. FIG. 5H illustrates a side view of gel 500 from FIG. 5G. In other embodiments, the one or more tabs/locking elements 505 may be formed on one or more sides of the gel 500, or as one continuous tab/lip/rib around the perimeter of the gel body 500 (as shown in FIG. 5A-5B).

In another embodiment, the gel 500 is configured in a predefined solidified shape and includes indentations in one or more surfaces. For example, the indentations may be a word or phrase. As seen in FIGS. 5A and 5B, the gel 500 includes the words “Gel Shot” indented on the top surface 520. In another embodiment, one or more indentations may be formed in the side wall 530. The indentation is another form of the locking element for connecting with a tab or rib from an ultrasound component (e.g., a reverse configuration than the embodiment above).

With reference to FIGS. 6A-6E, one embodiment of a gel adaptor 600 is shown in a variety of views. For example, FIG. 6A: top perspective view; FIG. 6B: left side view; FIG. 6C: right side view; FIG. 6D: bottom perspective view; and FIG. 6E: cross-section view of section A-A from FIG. 6B. The gel adaptor 600 is one type of device in which the solidified gel 500 can be inserted into and interlocked with using the locking element 505.

The gel adaptor 600 is configured to attach to the head of an ultrasound device that does not have a gel cavity for holding a piece of gel preconfigured with a solidified shape. Once attached, the adaptor 600 converts the ordinary flat head surface of the ultrasound device to a device with a gel cavity 605 that can receive and hold a preconfigured piece of gel (e.g., gel 140—FIGS. 1A-1D; gel 500—FIGS. 5A-5C). In another embodiment, an ultrasound device may be configured to include a gel cavity 605 and locking recess 625 as integral components of the device.

The adaptor 600 is configured with a top opening (gel cavity) 605 (FIG. 6A) and a bottom opening 610 (FIG. 6D). The housing of the adaptor 600 may be configured with a shape that corresponds to the shape of an ultrasound head so that the bottom opening 610 and sidewalls connect/attached to the ultrasound head. In that regard, the adaptor 600 may be configured with various internal shapes and edges 615 to contact surfaces of the ultrasound head for a better fit. In other embodiments, the internal portion of the adaptor 600 may include one or more lips 620 provide additional connection points. The top opening/cavity 605 is configured to generally match the shape of the preconfigured gel being used (e.g., preconfigured gel 500 (FIG. 5A-5C)). Other shapes may include oval, rectangular, or other polygonal shape.

With reference to FIG. 6E, the adaptor 600 is configured with a gel locking ring/recess 625 within the gel cavity 605. The locking recess 625 generally corresponds to the locking element 505 (e.g., ring-shaped) of the gel 500 and is configured to receive/connect with the locking element 505 of the solidified gel 500. Since the gel 500 is a flexible/malleable substance, the gel 500 can be pressed into the cavity 605 until the two shapes align. Thus, the locking element 505 inserts into the corresponding locking recess 615 to lock the gel 500 in the cavity 605. Accordingly, the gel 500 is held in the cavity 605.

Opening/Closing Adapter for Locking Ultrasound Medium

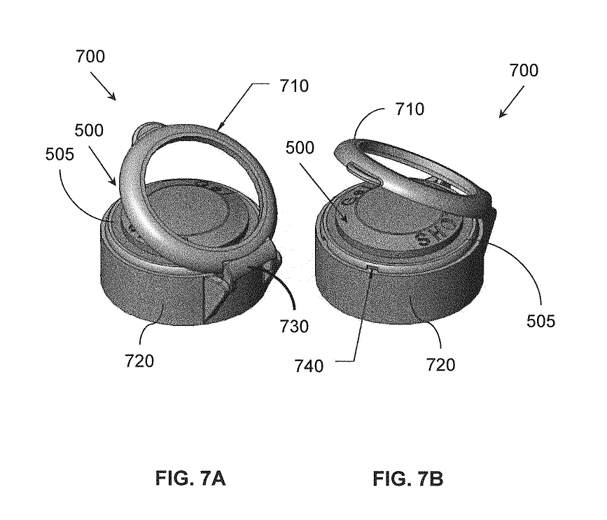

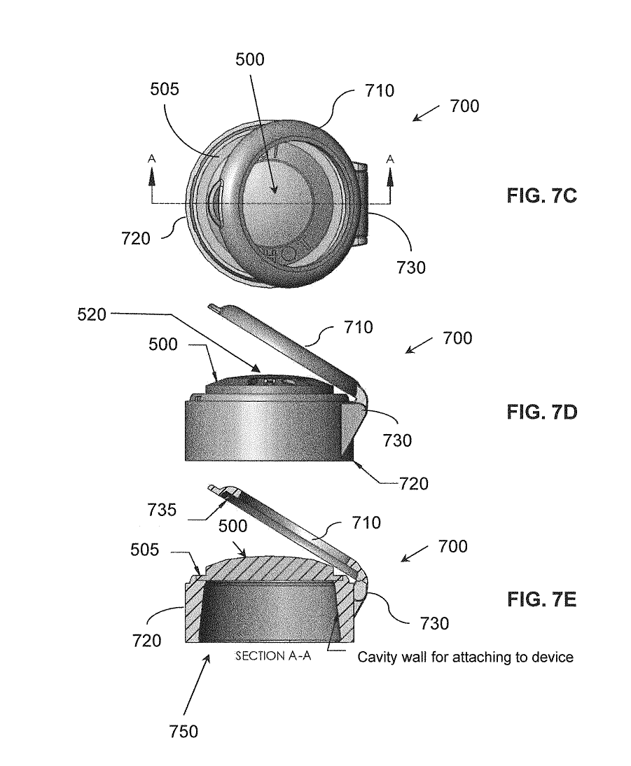

With reference to FIGS. 7A-7E, another embodiment of a gel adapter 700 is disclosed to demonstrate how the solidified gel 500 may be used and may be operatively connected to a device. The gel adaptor 700 is configured to attach to the head of an ultrasound device to create a gel receiving cavity for the gel 500 and a locking mechanism. In another embodiment, an ultrasound device may be configured to include similar components (e.g. the opening/closing top) that integral with the head of the device.

With reference to FIGS. 7A-7E, the adapter device 700 is illustrated in various views. FIG. 7A shows a perspective view of the adaptor 700 with a piece of the preconfigured gel 500 loaded within a receiving cavity. The preconfigured gel 500 is also shown in FIGS. 5A-5C and includes the locking element/ring 505 (e.g., protrusion, edge, or tab) that extends from the solidified gel 500. The adapter 700 includes a locking mechanism comprising an adapter top portion 710 and an adapter base 720. FIG. 7B shows a perspective view that is rotated from FIG. 7A. Both figures show the adaptor top 710 in an open position. FIG. 7C is a top view showing the top portion 710 open with the gel 500 inside. FIG. 7D is a side view showing the top portion open and the gel 500 loaded inside the adaptor 700. FIG. 7E is a cross-section view through line A-A (shown in FIG. 7C).

The adapter 700 includes a hinge 730 that connects the top portion 710 to the base 720 and allows the top portion 710 to move relative to the base 720 at the hinge 730. For example, the top 710 can be opened and unlocked to the top surface of the base 720, or be closed and attached/locked to the top surface of the base 720. The top portion 710 may include one or more latch tabs 735 that fit/snap into corresponding recesses 740 formed on the base 720 for locking the top 710 against the base 720 when the top is closed.

FIG. 7E shows the top portion 710 configured with edges that when closed against the base 720, overlap and press/clamp down against the gel locking element 505 to lock the gel 500 into the adapter 700. Thus the two opposing sides 510 and 515 of the locking element 505 (as seen in FIG. 5D) engage with the edges from top 710 and base 720 to secure the gel 500 (as seen in FIG. 7E). The top 710 may create a compression force against the locking element 505 of the gel 500 (and/or other parts of the gel 500) to secure the gel 500 when the top 710 is locked with the base 720.

When the top 710 is closed onto the base 720, the top 710 and base 720 are configured to create a gel cavity for the gel 500 and a locking recess in which the locking element 505 fits into. These components are similar to the gel cavity 605 and locking recess 625 of the adaptor device 600 shown in FIGS. 6A-6E, except that the adaptor device 700 is openable.

The top portion 710 includes an opening to allow the top surface 520 of the gel 500 to be exposed. When using the adapter 700 with an ultrasound device/applicator, the gel 500 is exposed to contact the skin of a patient. By opening the top portion 710, gel 500 can be removed from the adapter 700 and replaced with a new piece of gel by inserting the gel into the receiving cavity of the adapter 700. By closing the top portion 710 against the base 720, the gel 500 is locked into place since the edges of the top portion 710 overlap against the locking element 505 of the gel 500. For example, the top portion 710 overlaps and engages against surface 510 of the locking element 505 (see FIG. 5D).

The base 720 is formed with a mounting cavity 750 that is configured to correspond to the shape of an ultrasound head or applicator tip onto which the adapter 700 will be attached (e.g., pushed onto, locked, friction hold, snapped onto, etc). Thus, the adaptor 700 can be retrofitted onto an existing device head and convert the head to one that carries and uses replaceable pieces of preconfigured gel. Thus the adaptor 700 becomes the head portion of the ultrasound device. In another embodiment, an ultrasound device/probe may be configured with a head portion that includes the elements of the adaptor 700. Thus, the locking mechanism of the adaptor 700 may be integral with the ultrasound device.

In another embodiment, the top portion 710 is detachable from the base 720 and does not include the hinge 730. In this manner, the top portion 710 is a separate component from the adaptor base 720 where the top portion 710 is snapped on the top surface of the base 720 to lock in the gel 500. The top portion 710 is disconnected and removed from the base 720 to open and release the gel 500. In another embodiment, the top portion 710 is connected to the base 720 via one or more columns or posts. Thus rather than rotating along a hinge, the top portion 710 may move vertically up and down with the posts relative to the top surface of the base 720 to open or close the top portion 710. In one embodiment, the posts are configured to retract into the side walls of the adapter base 720.

Definitions

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

The term “conductive medium” is used to refer to a substance that is used during an ultrasound procedure that assists in coupling the ultrasound device/probe head or applicator tip to a subject (e.g., the skin of a patient or other surface) and conducts ultrasound energy. Typically, the conductive medium is ultrasound gel but other liquid-based substances, water-based substances, or oil-based substances can be used that are appropriate to function with an ultrasound device. Many liquid substances can form gels when a suitable thickener or gelling agent is added to their formula to change the viscosity. These substances may be preconfigured into a solidified state as an individual piece of conductive medium using a solidifying process suitable for the particular gel composition used (e.g., curing, heating, cooling, etc.). For example, a solidified state is any state in which the gel holds its shape and does not flow (may be flexible or firm). References to the term “gel” is intended to refer to any of these conductive media that is appropriate as an interface/coupling medium for ultrasound energy.

As previously stated, one purpose of the gel is to convey acoustic energy from the ultrasound head to the tissue of a patient without passing through air or passing through a minimal amount of air. Air (including air bubbles) can interfere or degrade the ultrasound signals.

While example systems, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described.

Thus, this application is intended to embrace alterations, modifications, and variations that fall within the scope of the appended claims.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use. See, Bryan A. Garner, A Dictionary of Modern Legal Usage 624 (2d. Ed. 1995).

What is claimed is:

1. An article of manufacture comprising an ultrasound conductive medium configured into a predefined shape and including a protruding portion that extends out from a surface of the ultrasound conductive medium.

2. The article of manufacture of claim 1, wherein the protruding portion is a locking element for securing the ultrasound conductive medium to a device.

3. The article of manufacture of claim 1, wherein the predefined shape is a solidified disk shape that includes a top surface, an opposing bottom surface, and a side surface therebetween; and
wherein the protruding portion is a tab that extends out from the side surface and is generally parallel to and extends out from the bottom surface, and wherein the tab is continuous around the perimeter of the solidified disk shape along the side surface; and
wherein the tab is configured to engage with a locking mechanism in an ultrasound device or adaptor to lock the ultrasound conductive medium to the ultrasound device.

4. The article of manufacture of claim 1, wherein the protruding portion is formed around a perimeter of the ultrasound conductive medium and is configured to secure the ultrasound conductive medium to an ultrasound device.

5. The article of manufacture of claim 1, wherein the ultrasound conductive medium includes two or more protruding portions that extend out from a side wall of the ultrasound conductive medium.

6. The article of manufacture of claim 1, wherein the ultrasound conductive medium is formed as a solidified substance that conducts ultrasound energy; and
wherein the predefined shape is a disk shape and wherein the protruding portion is a continuous tab extending around the perimeter of the disk shape.

7. The article of manufacture of claim 1, wherein the protruding portion is a tab that extends out from a side wall of the predefined shape, wherein the tab includes two opposing sides and wherein each side includes a surface.

8. A device comprising:
an ultrasound conductive substance formed into a solidified shape; and
a locking element formed in a side of the solidified shape, wherein the locking element is configured to secure the solidified shape to a component in an ultrasound device.

9. The device of claim 8, wherein the locking element is one or more tabs.

10. The device of claim 8, wherein the locking element extends out from the side of the solidified shape and includes two opposing sides.

11. The device of claim 8, wherein the locking element includes one or more protruding portions that extend out from the solidified shape that are configured to engage with a locking mechanism in the component of the ultrasound device.

12. The device of claim 8, wherein solidified shape is a disk shape; and
wherein the locking element is a ring-shaped tab that extends out from the side of the disk shape and continuously around a perimeter of the disk shape.

13. The device of claim 8, wherein the ultrasound conductive substance includes a gel-based substance that conducts ultrasound energy.

14. The device of claim 8, wherein the solidified shape includes a top surface and an opposing bottom surface, and wherein the locking element is a tab that extends generally parallel to and extending out from the bottom surface.

15. The device of claim 8, wherein the locking element includes one or more indentations formed in the solidified shape.

16. An article of manufacture, comprising:
an ultrasound conductive substance formed into a solidified shape, wherein the solidified shape includes a top surface, a bottom surface, and a side surface between the top surface and the bottom surface;
wherein the solidified shape is a disk-shape; and
one or more tabs extending out from the side surface of the disk-shape.

17. The article of manufacture of claim 16, wherein the one or more tabs includes a tab extending continuously around a perimeter of the solidified shape.

18. The article of manufacture of claim 16, wherein the one or more tabs are configured to interconnect with a component of an ultrasound device to secure the solidified shape to the ultrasound device.

19. The article of manufacture of claim 16, wherein the solidified shape is connected to a device, wherein the device comprises a gel receiving cavity and a recess formed in a wall of the gel receiving cavity; and
wherein the solidified shape is positioned within the gel receiving cavity and the one or more tabs are fitted within the recess formed in the wall of the gel receiving cavity.
